(19) United States
(12) Patent Application Publication
(10) Pub. No.: US 2021/0360864 A1
(43) Pub. Date: Nov. 25, 2021

(54) SYSTEM AND METHOD FOR PRESERVING CUT-FLOWERS

(71) Applicant: Chang Hoon LEE, Seoul (KR)

(72) Inventor: Chang Hoon LEE, Seoul (KR)

(21) Appl. No.: 17/028,765

(22) Filed: Sep. 22, 2020

(30) Foreign Application Priority Data
May 22, 2020 (KR) 10-2020-0061333
Sep. 16, 2020 (KR) 10-2020-0119506

(51) Int. Cl. A01G 5/06; B01D 53/26; A01N 3/00

(52) U.S. Cl. CPC A01G 5/06; A01N 3/00; B01D 53/265; B01D 53/261

(57) ABSTRACT

A system for preserving cut-flowers using an atmospheric pressure plasma generator according to an embodiment of the present application includes a container that preserves cut-flowers, an air filter that extracts at least some of moisture contained in air in the container and discharges the same, an air compressor that generates compressed air from the air from which the at least some of the moisture was removed by the air filter, and a plasma generator that generates active species through plasma discharge using the compressed air generated from the air compressor as a plasma discharge gas.

### CROSS-REFERENCE TO RELATED APPLICATION

This application is based upon and claims the benefit of priority from Korean Patent Application No. 10-2020-0061333, filed in the Korean Intellectual Property Office on May 22, 2020, and Korean Patent Application No. 10-2020-0119506, filed in the Korean Intellectual Property Office on Sep. 16, 2020, the entire contents of which are incorporated herein by reference.

### FIELD

The present disclosure relates to a system and method for preserving cut-flowers, and more specifically, to a system and method for preserving cut-flowers using an atmospheric pressure plasma generator.

### BACKGROUND

Plasma is an ionized gas such as electrons, neutral particles, or the like, and a part of the plasma gas has high energy, which can change a surface of a material. That is, the plasma may react directly with the surface of another material or react by elastic collision. The plasma generator mainly includes a tube which is configured to generate plasma with the compressed air or nitrogen gas colliding with high-frequency, high-voltage electric charges. In recent years, the use of atmospheric pressure plasma devices in place of low pressure or vacuum plasma is increasing. The atmospheric pressure plasma device maybe applied to various materials and substrates through a low-temperature process, and since a vacuum container or a vacuum evacuation device is not necessary, the treatment is fast and economical. In addition, when used, a deposition method using the atmospheric pressure plasma provides good adhesion and low deposition temperature, and thus, it is used in relatively various industries by utilizing the advantages such as reduction in deformation or denaturation that accompanies high temperature heating in the conventional surface treatment process, semiconductor process, and display process.

In general, ethylene oxide (EO) gas, ozone, low-temperature plasma, and the like may be used for disinfection and sterilization of biomaterials such as medical equipment or food. Among them, the medical sterilizer using plasma gas uses a hydrogen peroxide sterilization method, but there is a problem that a large amount of cost and space are required to purchase and operate the equipment. Meanwhile, the atmospheric pressure plasma generator generates, by discharge, various active species including ozone. Since these active species have a sterilization, disinfection or bleaching action, they can be used for sterilization or disinfection or air purification of various articles or biomaterials.

### SUMMARY

Embodiments disclosed herein provide a system and method for preserving cut-flowers, capable of controlling moisture and of antibacterial treatment inside a container for preserving cut-flowers, using a plasma generator and an air filter that generates compressed air.

A system for preserving cut-flowers using an atmospheric pressure plasma generator according to an embodiment of the present disclosure may include a container that preserves cut-flowers, an air filter that extracts at least some of moisture contained in air in the container and discharges the same, an air compressor generating compressed air from air from which at least some of the moisture was removed by the air filter; and a plasma generator that generates active species through plasma discharge using the compressed air generated from the air compressor as a plasma discharge gas.

According to an embodiment, the plasma generator may include a gas supply pipe through which the compressed air is supplied, and a nozzle part through which plasma gas containing the active species is discharged.

According to an embodiment, the system for preserving cut-flowers may further include a receptacle that accommodates the cut-flowers in the container, in which the receptacle may be manufactured using an antibacterial material.

According to an embodiment, the receptacle may be manufactured by injection molding a plastic containing a silica nanocomposite containing silver nanoparticles dispersed therein.

According to an embodiment, the system for preserving cut-flowers may further include: a humidity sensor that measures an amount of moisture contained in the air in the container, an active species sensor that measures a concentration of the active species in the container, and a controller that controls whether or not to operate the air filter based on the amount of moisture measured by the humidity sensor controls whether or not to operate at least one of the air compressor and the plasma generator based on the concentration of the active species measured by the active species sensor.

A method for preserving cut-flowers using an atmospheric pressure plasma generator according to an exemplary embodiment of the present disclosure may include extracting, by an air filter, at least some of moisture contained in air in a container in which the cut-flowers are preserved and discharging the same, generating, by the air filter, compressed air from the air from which the at least some of the moisture was removed by the air compressor, and generating, by a plasma generator, active species through plasma discharge by using the compressed air generated from the air compressor as a plasma discharge gas.

According to an embodiment, the method for preserving cut-flowers may further include discharging, by the plasma generator, plasma gas containing the active species into the container.

According to an embodiment, in the method for preserving cut-flowers, the container may further include a receptacle that accommodates the cut-flowers, in which the receptacle may be manufactured using an antibacterial material.

According to an embodiment, the receptacle may be manufactured by injection molding a plastic containing a silica nanocomposite containing silver nanoparticles dispersed therein.

According to an embodiment, the method for preserving cut-flowers may further include measuring, by a humidity sensor, an amount of moisture contained in the air in the container, measuring, by an active species sensor, a concentration of the active species in the container, controlling, by a controller, whether or not to operate the air filter based on the amount of moisture measured by the humidity sensor, and controlling, by the controller, whether or not to operate at least one of the air compressor and the plasma generator based on the concentration of active species measured by the active species sensor.

According to various embodiments of the present disclosure, it is possible to increase the freshness and preservation period of cut-flowers by removing moisture contained in air in a container in which cut-flowers are preserved to maintain a constant humidity.

In addition, according to various embodiments of the present disclosure, by generating a plasma gas having a sterilization or disinfection effect while maintaining a constant humidity of the air in the container in which the cut-flowers are preserved, the freshness and preservation period of the cut-flowers can be further increased, thereby improving the marketability of the cut-flowers.

Further, according to various embodiments of the present disclosure, by including a preservative in a cut-flower receptacle made of an antibacterial material, antibacterial and disinfection of the preservative are enabled and it is possible to increase the freshness and preservation period of the cut-flower.

The effects of the present disclosure are not limited to the effects described above, and other effects that are not mentioned above can be clearly understood to those skilled in the art based on the description provided below.

### DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 is a schematic diagram showing a system for preserving cut-flowers using an atmospheric pressure plasma generator according to an embodiment of the present disclosure;

FIG. 2 is a schematic diagram showing a system for preserving cut-flowers using an atmospheric pressure plasma generator according to another embodiment of the present disclosure;

FIG. 3 is a perspective view schematically showing a plasma generator according to an embodiment of the present disclosure;

FIG. 4 is a cross-sectional view of a tray for preserving cut-flowers according to an embodiment of the present disclosure;

FIG. 5 is a flowchart showing a method for preserving cut-flowers using an atmospheric pressure plasma generator according to an embodiment of the present disclosure; and

FIG. 6 is a flowchart showing a method for preserving cut-flowers using an atmospheric pressure plasma generator according to another embodiment of the present disclosure.

### DETAILED DESCRIPTION

Hereinafter, specific details for the practice of the present disclosure will be described in detail with reference to the accompanying drawings. However, in the following description, detailed descriptions of well-known functions or configurations will be omitted when it may make the subject matter of the present disclosure rather unclear.

In the accompanying drawings, the same or corresponding components are given the same reference numerals. In addition, in the following description of the embodiments, duplicate descriptions of the same or corresponding components may be omitted. However, even if descriptions of components are omitted, it is not intended that such components are not included in any embodiment.

The terms used in the present disclosure will be briefly described prior to describing the disclosed embodiments in detail. The terms used herein have been selected as general terms which are widely used at present in consideration of the functions of the present disclosure, and this may be altered according to the intent of an operator skilled in the art, conventional practice, or introduction of new technology. In addition, in a specific case, a term is arbitrarily selected by the applicant, and the meaning of the term will be described in detail in a corresponding description of the embodiments. Therefore, the terms used in the present disclosure should be defined based on the meaning of the terms and the overall contents of the present disclosure rather than a simple name of each of the terms.

In the present disclosure, the singular forms “a,” “an,” and “the” are intended to include the plural forms as well, unless the context clearly indicates the singular forms. Further, the plural forms are intended to include the singular forms as well, unless the context clearly indicates the plural forms.

In the present disclosure, when a portion is stated as “comprising (including)” a component, unless specified to the contrary, it intends to mean that the portion may additionally comprise (or include or have) another component, rather than excluding the same.

As used herein, the statement “A and/or B” means A, or B, or A and B.

Prior to describing the embodiments of the present disclosure, it is to be noted that the upper direction of the drawing may be referred to as “upper portion” or “upper side” of the configuration shown in the drawing, and the lower direction maybe referred to as “lower portion” or “lower side”. In addition, in the drawings, a portion between the upper and lower portions of the configuration shown in the drawings, or a portion other than the upper and lower portions may be referred to as “side portion” or “side”. Relative terms such as “upper portion” and “upper side” may be used to describe the relationship between components shown in the drawings, and the present disclosure is not limited by these terms.

In embodiments of the present disclosure, the left direction of the drawing may be referred to as “left” or “left side” of the configuration shown in the drawing, and the right direction thereof maybe referred to as “right” or “right side”. Relative terms such as “left” and “left side” may be used to describe the relationship between components shown in the drawings, and the present disclosure is not limited by these terms.

In the present disclosure, a direction toward the inner space of a structure may be referred to as “inside”, and a direction projecting into the open outer space maybe referred to as “outside”. Relative terms such as “inside” and “outside” may be used to describe the relationship between components shown in the drawings, and the present disclosure is not limited by these terms.

Throughout the description, when a portion is stated as being connected to another portion, it intends to include not only an example in which the portions are directly connected, but also an example in which the portions are connected while having another component disposed therebetween.

In the present disclosure, the term “part” or “portion” or “module” means a mechanical or hardware component, a software component, or a combination thereof, and a “unit” or “module” may be configured to perform a specific role or function. However, it does not mean that “unit” or “module” is limited to a mechanical component or hardware or software. The “unit” or “module” may be configured to be in an addressable storage medium or to execute one or more processors. Accordingly, as an example, the “unit” or “module” includes elements such as software elements, object-oriented software elements, class elements, and task elements, processes, functions, attributes, procedures, subroutines, program code segments, drivers, firmware, micro-codes, circuits, data, database, data structures, tables, arrays, and variables. Functions provided in the components and the “units” or “modules” described in the present disclosure may be combined as a smaller number of components and “units” or “modules”, or further divided into additional components and “units” or “modules”.

In the present disclosure, “system” may mean a mechanical device or an electromechanical device including one or more plasma generators, air compressors, air filters, computing devices, containers, and the like, but is not limited thereto.

Advantages and features of the disclosed embodiments and methods of accomplishing the same will be apparent by referring to embodiments described below in connection with the accompanying drawings. However, the present disclosure is not limited to the embodiments disclosed below, and may be implemented in various different forms, and the present embodiments are merely provided to make the present disclosure complete, and to fully disclose the scope of the invention to those skilled in the art to which the present disclosure pertains.

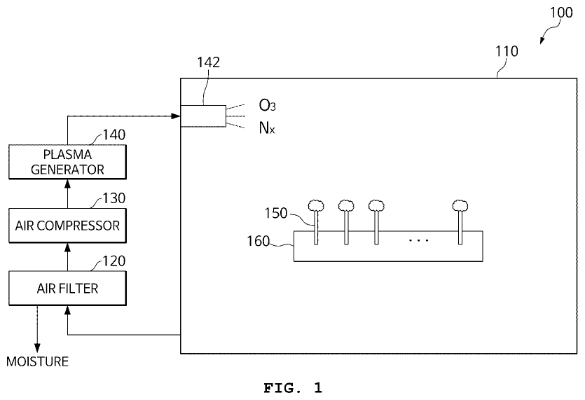

FIG. 1 is a schematic diagram showing a system 100 for preserving cut-flowers using an atmospheric pressure plasma generator according to an embodiment of the present disclosure.

As shown, the system 100 for preserving cut-flowers may include a container 110 in which one or more cut-flowers 150 are preserved, an air filter 120 that extracts at least some of moisture contained in air in the container 110 and discharges the same, an air compressor 130 that generates compressed air from the air from which at least some of the moisture was removed by the air filter 120, and a plasma generator 140 that generates active species through plasma discharge using the compressed air generated from the air compressor 130 as a plasma discharge gas.

The container 110 may be used for the purpose such as preservation or transport of the cut-flowers 150 for a certain period of time until the cut-flowers 150 from the production site are delivered to the purchaser. Accordingly, the container 110 may be installed in a fixed position or configured to be detachable from transport equipment such as a vehicle or a ship.

In addition, one or more supports 160 for the cut-flowers 150 to be mounted thereon may be disposed in the container 110. The support 160 may be configured to mount and preserve the cut-flowers 150. In an embodiment, the support 160 may be in a box shape with an empty space inside, and may include a plurality of through holes through which the cut-flowers 150 can be inserted into the support 160.

The system 100 for preserving cut-flowers may be configured such that an air filter 120 is connected to one side of the container 110, thus allowing air in the container 110 to be introduced into the air filter 120. The air filter 120 may be configured to remove at least some of the moisture contained in the air introduced from the container 110. With this configuration, the air in the container 110 may be managed at a constant humidity. In an embodiment, the air filter 120 may include a cooling or dry dehumidifier. For example, the dry dehumidifier may be configured to adsorb the moisture contained in the air by using a desiccant such as silica gel, alumina gel, or molecular sieves, which are porous materials. In another example, the cooling dehumidifier may be configured to suck in air with a fan and then pass it through a cooling device where refrigerant is used, thereby using a characteristic of the air that turns into water as the temperature of the air is decreased to the dew point.

The air, from which moisture is removed by the air filter 120, may be introduced into the air compressor 130. The air compressor 130 may compress the introduced air to a constant pressure and provide it to the plasma generator 140. In an embodiment, the air compressor 130 may include a reciprocating or piston type, a rotary screw type, or a turbo or centrifugal type compressor, but is not limited thereto, and may include any type of compressor that has an appropriate capacity to provide air of a constant pressure as required by the plasma generator 140.

The plasma generator 140 may generate active species including ozone (O₃) through plasma discharge using the introduced compressed air as the plasma discharge gas. In an embodiment, the plasma generator 140 may be an atmospheric pressure plasma generator. The plasma gas including the active species generated by the plasma generator 140 may be discharged into the container through a nozzle part 142 attached inside the container 110. The harmful bacteria or harmful substances contained in the air inside the container 110 may be removed by the plasma gas including the active species discharged as described above, and accordingly, the freshness or preservation period of the cut-flowers 150 may be increased.

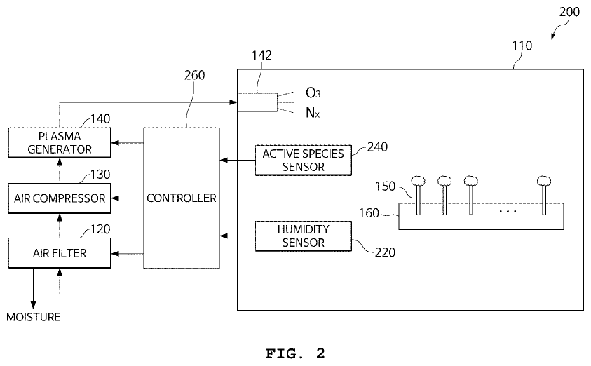

FIG. 2 is a schematic diagram showing a system 200 for preserving cut-flowers using an atmospheric pressure plasma generator according to another embodiment of the present disclosure. Among the configurations disclosed in FIG. 2, a description of the configurations corresponding to those of FIG. 1 will be omitted.

As shown, the system 200 for preserving cut-flowers may include the container 110, the air filter 120, the air compressor 130, the plasma generator 140, a humidity sensor 220 and an active species sensor 240 installed in the container 110, and a controller 260 that controls one or more of the air filter 120, the air compressor 130, and the plasma generator 140 so that the air in the container 110 has a constant humidity and concentration of active species. In the system 200 for preserving cut-flowers, the controller 260 that received a signal from the humidity sensor 220 and/or the active species sensor 240 controls the air filter 120, the air compressor 130, and/or the plasma generator 140, thereby maintaining the humidity and the concentration of active species of the air inside the container 110 within a predetermined numerical range.

In an embodiment, the humidity sensor 220 may be installed inside the container 110. The humidity sensor 220 installed inside the container 110 maybe configured to measure the amount of moisture or humidity of the air inside the container 110. Examples of the humidity sensor 220 may include an electric resistive type or electric capacitive type humidity sensor capable of outputting an electrical signal according to humidity, but it is not limited thereto. The humidity sensor 220 may serve to check, in real time, whether or not the amount of moisture or humidity of the air inside the container 110 is maintained within the predetermined numerical range. The humidity sensor 220 may transmit, to the controller 260, an electrical signal determined according to the amount of moisture or humidity contained in the air inside the container 110.

In an embodiment, the active species sensor 240 may be installed inside the container 110. The active species sensor 240 installed inside the container 110 may be configured to measure the concentration of the active species (e.g., O₃, Nₓ, and the like) of the air inside the container 110. Examples of the active species sensor 240 may include a chemical sensor capable of outputting an electrical signal by changing electrical conductivity according to adsorption or desorption of active species in gaseous state, but it is not limited thereto. The active species sensor 240 may serve to check, in real time, whether or not the concentration of active species of the air inside the container 110 is maintained at a concentration equal to or higher than a reference value. The active species sensor 240 may transmit, to the controller 260, an electrical signal determined according to the presence or absence of the active species contained in the air inside the container 110 or the concentration of the active species.

The controller 260 may receive an electrical signal transmitted from the humidity sensor 220 and/or the active species sensor 240, and determine whether the humidity and/or the concentration of the active species contained in the air inside the container 110 is equal to or greater than or less than a reference value. The controller 260 may control the operating time and intensity of one or more of the air filter 120, the air compressor 130, and the plasma generator 140 according to the humidity or the concentration of the active species in the container 110 determined as described above. For example, when determining that the humidity of the air inside the container 110 is equal to or greater than the reference value based on the electrical signal of the humidity sensor 220, the controller 260 may operate the air filter 120 or increase the operating time and/or intensity of the air filter 120. In addition, when determining that the concentration of active species of the air inside the container 110 is equal to or less than the reference value based on the electrical signal of the active species sensor 240, the controller 260 may operate the air compressor 130 and/or the plasma generator 140, or increase the operating time and/or intensity of the air compressor 130 and/or the plasma generator 140.

In an embodiment, the control of the operation of the air filter 120 by the controller 260 as described above, and the control of the operation of the air compressor 130 and/or the plasma generator 140 by the controller 260 may be executed sequentially. For example, the controller 260 may control the operation of the air filter 120 and then control the operation of the air compressor 130 and/or the plasma generator 140, and may also repeat these two steps of control of the operation. In another embodiment, the control of the operation of the air filter 120 by the controller 260 and the control of the operation of the air compressor 130 and/or the plasma generator 140 by the controller 260 may be executed in parallel and simultaneously. In still another embodiment, either the control of the operation of the air filter 120 by the controller 260 or the control of the operation of the air compressor 130 and/or the plasma generator 140 by the controller 260 may be executed.

As described above, by controlling the operation of the air filter 120 by the controller 260 and controlling the operation of the air compressor 130 and/or the plasma generator 140 by the controller 260, the concentration of moisture and active species contained in the air inside the container 110 is maintained within an appropriate range, so that the freshness and preservation period of the cut-flowers 150 may be increased.

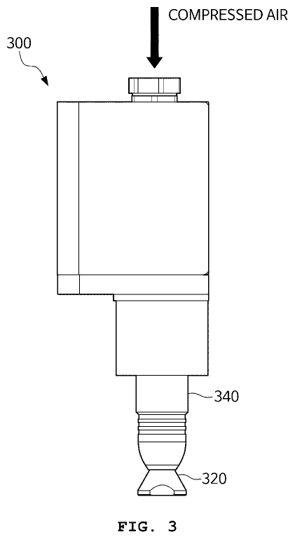

FIG. 3 is a perspective view schematically showing a plasma generator 300 according to an embodiment of the present disclosure. The plasma generator 300 shown in FIG. 3 may be used as the plasma generator 140 of the systems 100 and 200 for preserving cut-flowers shown in FIGS. 1 and 2, for example.

In an embodiment, the plasma generator 300 may be an atmospheric pressure plasma generator. In this case, the plasma generator 300 may include a generator, a high voltage transformer, electrodes for generating plasma discharge, and the like so as to be operated in a normal room temperature/atmospheric pressure environment. Specifically, the plasma generator 300 may include a nozzle part 320 through which plasma is discharged, a gas supply pipe (not shown) where the nozzle part 320 is attached to or detached from one side and a working gas (or compressed air) is supplied from the other side, and a body portion 340 to which a cable or the like connected to a high voltage transformer (not shown) is detached.

In an embodiment, the high frequency high voltage generated by the high voltage transformer is applied to the electrodes installed inside the body portion 340, and a high frequency discharge in the form of an electric arc is generated between the electrodes by the applied voltage. As described above, with the electric arc being generated inside the body portion 340, the working gas contacting the electric arc is converted into the plasma state. The plasma beam generated in the body portion 340 is discharged through an opening of the nozzle part 320.

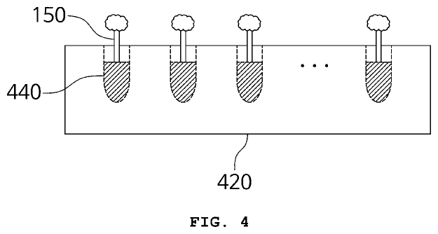

FIG. 4 is a cross-sectional view of a tray 420 for preserving cut-flowers according to an embodiment of the present disclosure. The tray 420 for preserving cut-flowers shown in FIG. 4 may be used as the support 160 installed inside the container 110 shown in FIGS. 1 and 2, for example.

One or more trays 420 for preserving cut-flowers may be disposed in the container (e.g., reference numeral 110 in FIG. 1). The tray 420 for preserving cut-flowers may be configured to mount and preserve the cut-flowers 150. As shown, the tray 420 for preserving cut-flowers may be in a box shape having a rectangular cross section, but is not limited thereto.

In an embodiment, the tray 420 for preserving cut-flowers may include a plurality of cut-flower receptacles 440 receiving therein a preservative including nutrients and moisture for maintaining the freshness of the cut-flowers 150. Bacteria may grow in the cut-flower receptacles 440 due to nutrients injected into the preservative for freshness of flowers. The cut-flowers 150 accommodated in the preservative with the propagating bacteria may have a sharp decrease in freshness and preservation period. To solve this problem, the cut-flower receptacles 440 may be manufactured with an antibacterial material such as a silver nanomaterial. For example, the cut-flower receptacles 440 may be molded by injecting a plastic containing a silica nanocomposite containing silver nanoparticles dispersed therein. By accommodating the preservative in the cut-flower receptacles 440 made of antibacterial material, antibacterial and disinfection of the preservative may be enabled.

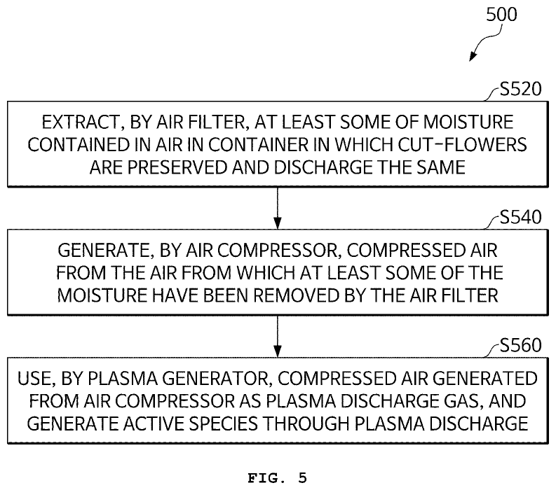

FIG. 5 is a flowchart showing a method 500 for preserving cut-flowers using an atmospheric pressure plasma generator according to an embodiment of the present disclosure.

The method 500 for preserving cut-flowers may be initiated by an air filter extracting and discharging at least some of the moisture contained in the air in the container in which the cut-flowers are preserved (S520). In an embodiment, referring to FIG. 1, the air filter 120 may be connected to one side of the container 110 such that air in the container 110 can be introduced into the air filter 120. The air filter 120 may be configured to remove at least some of the moisture contained in the air introduced from the container 110. With this configuration, the air in the container 110 may be managed at a constant humidity.

Next, by the air compressor, compressed air may be generated from the air from which at least some of the moisture have been removed by the air filter (S540). In an embodiment, referring to FIG. 1, the air, from which moisture is removed by the air filter 120, maybe introduced into the air compressor 130. The air compressor 130 may compress the introduced air to a constant pressure and provide it to the plasma generator 140.

The plasma generator may use the compressed air generated from the air compressor as the plasma discharge gas, and generate active species through plasma discharge (S560). In an embodiment, referring to FIG. 1, the plasma generator 140 may generate active species including ozone (O₃) through plasma discharge using the introduced compressed air as the plasma discharge gas. The plasma gas including the active species generated by the plasma generator 140 may be discharged into the container 110 through a nozzle part 142 attached inside the container 110. The harmful bacteria or harmful substances contained in the air inside the container 110 may be removed by the plasma gas including the active species discharged as described above, and accordingly, the freshness or preservation period of the cut-flowers 150 may be increased.

Additionally, it may be prepared so that a plurality of cut-flower receptacles are accommodated in the container and the preservative containing nutrients and moisture for maintaining the freshness of the cut-flowers may be accommodated in the cut-flower receptacles. In this case, the cut-flower receptacles may be molded by injecting a plastic containing a silica nanocomposite containing silver nanoparticles dispersed therein.

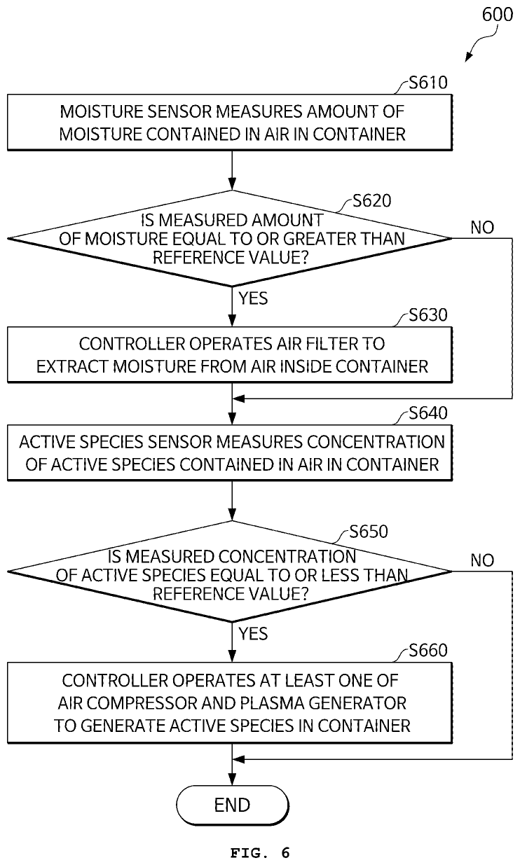

FIG. 6 is a flowchart showing a method 600 for preserving cut-flowers using an atmospheric pressure plasma generator according to another embodiment of the present disclosure.

The method 600 for preserving cut-flowers may be initiated by a moisture sensor measuring an amount of moisture contained in the air in the container (S610) . In an embodiment, referring to FIG. 2, the humidity sensor 220 installed inside the container 110 may measure the amount of moisture or humidity contained in the air inside the container 110. In addition, the humidity sensor 220 may transmit, to the controller 260, an electrical signal determined according to the amount of moisture or humidity contained in the air inside the container 110.

The controller may determine whether or not the measured amount of moisture is equal to or greater than the reference value (S620). In an embodiment, referring to FIG. 2, the controller 260 may determine whether or not the amount of moisture or humidity of the air inside the container 110 is equal to or greater than the reference value, based on the electrical signal from the humidity sensor 220.

When the amount of moisture measured in step S620 is determined to be equal to or greater than the reference value, the controller may operate the air filter to extract moisture from the air inside the container (S630). In an embodiment, referring to FIG. 2, when determining that the humidity of the air inside the container 110 is equal to or greater than the reference value based on the electrical signal of the humidity sensor 220, the controller 260 may operate the air filter 120 or increase the operating time and/or intensity of the air filter 120. As described above, the air filter 120 may manage the air in the container 110 at a constant humidity by removing at least some of moisture contained in the air introduced from the container 110.

Next, the active species sensor may measure the concentration of the active species contained in the air in the container (S640). In an embodiment, referring to FIG. 2, the active species sensor 240 installed inside the container 110 may transmit, to the controller 260, an electrical signal determined according to the presence or absence of the active species contained in the air inside the container 110 or the concentration of the active species.

The controller may determine whether or not the measured concentration of active species is equal to or less than a reference value (S650). In an embodiment, referring to FIG. 2, the controller 260 may determine whether or not the concentration of the active species in the air inside the container 110 is equal to or less than the reference value based on the electrical signal from the active species sensor 240.

When the concentration of the active species measured in step S650 is determined to be equal to or less than the reference value, the controller may operate at least one of the air compressor and the plasma generator to generate the active species in the container (S660). In an embodiment, referring to FIG. 2, when determining that the concentration of active species of the air inside the container 110 is equal to or less than the reference value based on the electrical signal of the active species sensor 240, the controller 260 may operate the air compressor 130 and/or the plasma generator 140, or increase the operating time and/or intensity of the air compressor 130 and/or the plasma generator 140. The harmful bacteria or harmful substances contained in the air inside the container 110 may be removed by the plasma gas including the active species discharged as described above, and accordingly, the freshness or preservation period of the cut-flowers 150 may be increased.

In an embodiment, the steps (S610 to S630) of controlling the operation of the air filter by the controller as described above and the steps (S640 to S660) of controlling the operation of the air compressor and/or the plasma generator by the controller may be executed sequentially. For example, after executing steps S610 to S630, steps S640 to S660 may be executed, and also, these steps may be repeatedly executed. In another embodiment, the steps (S610 to S630) of controlling the operation of the air filter by the controller and the steps (S640 to S660) of controlling the operation of the air compressor and/or the plasma generator by the controller may be executed in parallel and simultaneously. In still another embodiment, either the steps (S610 to S630) of controlling the operation of the air filter by the controller, or the steps (S640 to S660) of controlling the operation of the air compressor and/or the plasma generator by the controller may be executed.

Additionally, it may be prepared so that a plurality of cut-flower receptacles are accommodated in the container and the preservative containing nutrients and moisture for maintaining the freshness of the cut-flowers may be accommodated in the cut-flower receptacles. In this case, the cut-flower receptacles may be molded by injecting a plastic containing a silica nanocomposite containing silver nanoparticles dispersed therein.

The preferred embodiments of the present invention described above are disclosed for purposes of illustration, and those skilled in the art with ordinary knowledge of the present invention will be able to make various modifications, changes and additions within the spirit and scope of the present invention, and such modifications, changes and additions should be construed to be included in a scope of the claims.

When those skilled in the art to which the present invention belongs, various substitutions, modifications, and changes are possible within the scope of the technical spirit of the present invention, and thus the present invention is not limited by the embodiments described above and the accompanying drawings.

What is claimed is:

1. A system for preserving cut-flowers using an atmospheric pressure plasma generator, comprising:
a container that preserves cut-flowers;
an air filter that extracts at least some of moisture contained in air in the container and discharges the same;
an air compressor that generates compressed air from the air from which the at least some of the moisture was removed by the air filter; and
a plasma generator that generates active species through plasma discharge using the compressed air generated from the air compressor as a plasma discharge gas.

2. The system for preserving cut-flowers of claim 1, wherein the plasma generator includes:
a gas supply pipe through which the compressed air is supplied; and
a nozzle part through which plasma gas containing the active species is discharged.

3. The system for preserving cut-flowers of claim 1, further comprising a receptacle that accommodates the cut-flowers in the container, wherein the receptacle is manufactured using an antibacterial material.

4. The system for preserving cut-flowers of claim 3, wherein the receptacle is manufactured by injection molding a plastic containing a silica nanocomposite containing silver nanoparticles dispersed therein.

5. The system for preserving cut-flowers of claim 1, further comprising:
a humidity sensor that measures an amount of moisture contained in the air in the container;
an active species sensor that measures a concentration of the active species in the container; and
a controller that controls whether or not to operate the air filter based on the amount of moisture measured by the humidity sensor, and controls whether or not to operate at least one of the air compressor and the plasma generator based on the concentration of the active species measured by the active species sensor.

6. A method for preserving cut-flowers using an atmospheric pressure plasma generator, comprising:
extracting, by an air filter, at least some of moisture contained in air in a container in which the cut-flowers are preserved and discharging the same;
generating, by the air filter, compressed air from the air from which the at least some of the moisture was removed by the air compressor; and
generating, by a plasma generator, active species through plasma discharge by using the compressed air generated from the air compressor as a plasma discharge gas.

7. The method for preserving cut-flowers of claim 6, further comprising discharging, by the plasma generator, plasma gas containing the active species into the container.

8. The method for preserving cut-flowers of claim 6, wherein the container includes a receptacle, wherein the receptacle accommodates the cut-flowers and is manufactured using an antibacterial material.

9. The method for preserving cut-flowers of claim 8, wherein the receptacle is manufactured by injection molding a plastic containing a silica nanocomposite containing silver nanoparticles dispersed therein.

10. The method for preserving cut-flowers of claim 6, further comprising:
measuring, by a humidity sensor, an amount of moisture contained in the air in the container;
measuring, by an active species sensor, a concentration of the active species in the container;
controlling, by a controller, whether or not to operate the air filter based on the amount of moisture measured by the humidity sensor; and
controlling, by the controller, whether or not to operate at least one of the air compressor and the plasma generator based on the concentration of active species measured by the active species sensor.
